(19) United States
(12) Patent Application Publication
(10) Pub. No.: US 2010/0125253 A1
(43) Pub. Date: May 20, 2010

(54) Dual-tip Catheter System for Boring through Blocked Vascular Passages

(75) Inventors: William John Olson, Menlo Park, CA (US); Charles McNall, Belmont, CA (US)

Correspondence Address: SHAY GLENN LLP, 2755 CAMPUS DRIVE, SUITE 210, SAN MATEO, CA, 94403, US

(73) Assignee: Avinger, Santa Clara, CA (US)

(21) Appl. No.: 12/272,697

(22) Filed: Nov. 17, 2008

(51) Int. Cl. A61M 25/01; A61B 17/22

(52) U.S. Cl. 604/267; 606/159

(57) ABSTRACT

A dual-tip catheter equipped with both a rotating cutting bit and a cavity for a probing guidewire tip for passage through chronic total occlusions or other refractory atherosclerotic plaque from diseased arteries. The catheter's guidewire tip and rotating cutting bit reside safely within an outer protective catheter sheath head when not in use, and alternately either the cutting bit or the guidewire tip will protrude out of the same distal opening of the sheath head as needed. By first drilling through refractory occlusions, and then inserting a guidewire tip into the occlusion opening formed by the cutting bit, the catheter can force the guidewire tip past the occlusion. After the guidewire tip has been fully inserted, the dual-tip catheter can be withdrawn, and an alternate catheter inserted and directed up the guidewire to the occlusion site, where the alternate catheter can perform further occlusion enlargement or therapeutic activity.

### BACKGROUND

A number of vascular diseases, such as coronary artery disease and peripheral vascular disease, are caused by the build-up of fatty atherosclerotic deposits (plaque) in the arteries. These deposits limit blood flow to the tissues that are supplied by that particular artery. Risk factors for this type of disease include advanced age, diabetes, high blood pressure, obesity, history of smoking, and high cholesterol or triglycerides.

When these deposits build up in the arteries of the heart, the problem is called coronary artery disease (CAD). When these deposits build up in the arteries of a limb, such as a leg, the condition is called peripheral artery disease (PAD). Symptoms of CAD—angina, heart disease, and heart attacks, are well known. Symptoms of PAD can include pain on walking, and wounds that do not heal. If PAD is not treated, it can eventually produce critical limb ischemia (CLI), gangrene, and loss of limb. Roughly 30% of the population over the age of 70 suffers from PAD.

When the plaque builds up to the point where an artery is totally occluded, the obstruction is referred to as a Chronic Total Occlusion (CTO). CTOs can confound the treatment of CAD, because the sudden loss of heart muscle can lead to sudden death. A CTO that occludes the peripheral arteries for PAD patients is also extremely serious. PAD patients that suffer from a CTO often enter a downward spiral towards death. Often the CTO in a peripheral artery results in limb gangrene, which requires limb amputation to resolve. The limb amputation in turn causes other complications, and roughly half of all PAD patients die within two years of a limb amputation.

For both CAD and advanced PAD, prompt treatment of such blockages is thus essential. Here, less invasive angioplasty or atherectomy procedures have many advantages. In these procedures, a catheter is inserted into the diseased artery and threaded to the blocked region. There the blockage may be either squeezed into a hopefully more open position by pressure from an inflated catheter balloon (balloon angioplasty), the blocked region may be kept open by a stent, or alternatively a physician may use a catheter to surgically remove the plaque from the inside of the artery (atherectomy).

As an example, for the treatment of PAD, atherectomy devices such as the Fox Hollow (now ev3) SilverHawk™ catheter (U.S. Pat. No. 6,027,514), are often used. These catheters may be threaded (usually with the aid of a guidewire) up the artery to a blocked region. There, the physician will usually position the catheter to make multiple passes through the blocked region of the artery, each time shaving a way a ribbon of plaque. The shaved ribbons of plaque are stored in the hollow nose of the device. By making multiple passes, the plaque may be substantially reduced, blood circulation may be restored to the limb, and the limb in turn saved from amputation.

In order to effectively treat the plaque, however, most modern catheters need to be threaded past the blocked region of the artery. This is because the active portions of most catheters, which are used to treat the blockage, are usually located on the side of the catheter, rather than on the tip of the catheter. This is due to simple mechanical necessity. The tip of the catheter must have a very small surface area, and thus is able to treat only a very small portion of the diseased artery. By contrast, the side of the catheter has a much larger surface area, and the catheter side thus conforms nicely to the sides of the diseased artery. Thus stents, balloons, atherectomy cutting tools, etc., are usually mounted on the sides of the catheter. The catheter must be threaded past the blocked portion of the artery in order to function properly.

When the artery is only partially blocked by plaque, the catheter can usually be maneuvered past the obstruction, and the active portions of the catheter can thus be brought into contact with the diseased portion of the artery. However when the artery is totally blocked, as is the case with a CTO, this option is no longer possible. The tip of the catheter encounters the obstruction, and further forward motion is blocked.

Simply trying to force a typical catheter past the obstruction usually isn't possible. The obstructions are typically composed of relatively tough fibrous material, which often also includes hard calcium deposits as well. Often, when physicians attempt to force guidewires or catheters past such obstructions, the guidewire or catheter device may instead exit the artery and enter the lumen outside the artery. This further damages the artery, further complicates the procedure, and decreases the chance of success. As previously discussed, the consequences of such procedure failures have a high mortality rate. Thus improved methods to allow catheters and guidewires to more readily penetrate through hardened plaque and CTO are thus of high medical importance.

A good summary of the present state of the art may be found in an article by Aziz and Ramsdale, “Chronic total occlusions—a stiff challenge requiring a major breakthrough: is there light at the end of the tunnel?” Heart 2005; 91; 42-48.

Previous attempts to produce devices for cutting through hardened plaque include U.S. Pat. No. 5,556,405 to Lary, U.S. Pat. No. 6,152,938 to Curry, and U.S. Pat. No. 6,730,063 to Delaney et. al.

U.S. Pat. No. 5,556,405 teaches an incisor catheter which features a bladed head stored in a catheter housing, which contains a number of slits though which the blades protrude. The blade is activated by a push-pull catheter. When the push-pull catheter is pushed, the bladed head protrudes through the slits in the housing, and the blade thus comes into contact with hardened plaque material. The blade does not rotate, but rather delivers linear cuts.

U.S. Pat. No. 6,152,938 teaches a general purpose catheter drilling device for opening a wide variety of different blocked (occluded) tubes. The device anchors the tip of the drill head against a face of the occlusion, and partially rotates the drill head using a rein attached to the drill head so that the drill head faces at an angle.

U.S. Pat. No. 6,730,063 teaches a catheter device for chemically treating calcified vascular occlusions. The device is a fluid delivery catheter that delivers acidic solutions and other fluids to calcified plaque with the objective of chemically dissolving the calcified material.

Several catheter devices for traversing CTO obstructions are presently marketed by Cordis Corporation, FlowCardia Technology, Kensey Nash Corporation, and other companies. Cordis Corporation, a Johnson and Johnson Company, produces the Frontrunner® XP CTO catheter (formerly produced by LuMend Corporation). This catheter, discussed in U.S. Pat. No. 6,800,085 and other patents, has a front “jaw” that opens and closes as it traverses the catheter. The jaw itself does not cut, but rather attempts to pry open the CTO as the catheter passes.

Other catheter devices use various forms of directed energy to traverse CTOs. For example, FlowCardia Technology, Sunnyvale Calif., produces the Crosser system, taught in U.S. Pat. No. 7,297,131 and other patents. This system uses an ultrasonic transducer to deliver energy to a non-cutting catheter head. This catheter head itself has a relatively small diameter and does not have any blades. Rather, the head, through rapid (ultrasonic) vibration is able to push its way through a variety of different occlusions.

Kensey Nash Corporation, Exton Pa. (formerly Intraluminal Therapeutics, Inc.), produces the Safe-Cross CTO system. This system, taught in U.S. Pat. Nos. 6,852,109 and 7,288,087, uses radiofrequency (RF) energy. The catheter itself is also directed in its movement by an optical (near-infrared light) sensor which can sense when the tip of the catheter is near the wall of the artery. The optical sensor tells the operator how to steer the catheter, and the RF ablation unit helps the operator ablate material and cross occluded regions.

Although ingenious, the success rates with these devices still leave much to be desired. According to Aziz, the best reported success rates of overcoming CTOs with prior art devices range from 56% to 75%. Aziz further teaches that the average success rates are only in the 50-60% range. Given the huge negative impact that unsuccessfully cleared CTO's, have on patient morbidity and mortality, clearly further improvement is desirable.

An additional problem with these prior art CTO clearing devices is that simply cutting a small channel though the CTO may not be sufficient to totally resolve the medical problem. Occasionally, the device that traverses the CTO should also remove (debulk) a substantial portion of the occlusion. This is because as previously discussed, removal of a substantial portion of the occlusion may be required in order to allow catheters with side mounted stents, balloons, and atherectomy cutting tools to get access to the damaged portions of the artery and make more lasting repairs. Thus improved CTO “unclogging” devices that can do the more substantial amount of CTO debulking required to allow other types of catheters to pass are also desirable.

Thus there remains a need for devices that can effectively traverse CTOs and remove more substantial amounts of hardened or calcified plaque. Such devices would enable stents and other devices, such as SilverHawk atherectomy catheters, balloon catheters, etc. to be more successfully used in high occlusion situations. This in turn should lead to improved patient outcomes and a reduction in patient morbidity and mortality.

### INVENTION FIGURES

FIG. 1 shows an overview of the catheter device including the handle, the catheter, and the catheter sheath head and internal cutting bit. In this figure, a guidewire is also shown inserted into a cavity in the sheath head.

FIG. 2 shows various alternate configurations of the interior of the catheter sheath head. In one configuration, the guidewire and the cutting bit drive cable travel though separate lumens in the catheter, and the motion of the cutting bit may be guided or constrained by a front annular device and a rear bezel. In a second configuration, the guidewire and cutting bit drive cable travel through separate lumens, but the motion of the cutting bit may be otherwise not constrained. In a third configuration, the guidewire and the cutting bit drive cable travel though a common catheter lumen.

FIG. 3 shows various cross sections through the catheter tube and the catheter head, and also shows various embodiments of the invention.

FIG. 4 shows how the cutting bit and the guidewire tip may be alternately extended and retracted through the opening at the distal tip of the catheter head.

FIG. 5 shows use of the catheter in first cutting through the tough outer layer of a CTO obstruction, and then extending the guidewire through the CTO to the other side.

FIG. 6 shows how a the cutting bit catheter may then be removed, and a different balloon catheter or other device may then be introduced up the guidewire to the CTO, where the different catheter then can be used to further treat the CTO.

### DETAILED DESCRIPTION

The invention teaches a novel catheter for creating a passage through refractory material, such as chronic total occlusions, refractory atherosclerotic plaque, gallstones, kidney stones, etc., from diseased arteries, veins, or other body lumens. In one embodiment, the catheter has a rotating cutting bit designed to reside safely within an outer protective sheath head when not in use, and this sheath head may be mounted on the distal end of the catheter, and this sheath head will be at least partially hollow, and contain a distal opening.

Depending upon the angle and nature of the cutting bit's protruding blades, the blades may either be designed to simply cut thorough the occluding material, without actually dislodging the occluding material from the body lumen, or alternatively the blades may be designed to both cut through the occluding material, and sever its link to the body lumen, thereby dislodging the occluding material from the body lumen. In this case, the cutting bit can act to actually remove (debulk) a substantial portion of the occlusion

The interior of the outer protective sheath head may optionally contain a second surface complementary to the cutting bit. This surface may contain groves, slots, or annular openings. The cutting bit may optionally contain a first surface containing protruding blades or projections that fit into these groves, slots, or annular openings. Application of torque to an inner torque communicating connector (such as a catheter or tube, cable, wire or coil, or any torque communicating mechanism attached to the cutting bit) applies spin to the cutting bit. In one embodiment, the force of the second surface of an inner mechanism inside the sheath head against the first surface of the cutting bit's protruding blades or projections may then advance the cutting head forward from the interior of the protective sheath and outward through a distal opening in the sheath head. By reversing the direction of the torque, this process may be reversed. In an alternative embodiment, the cutting bit's protruding blades may spin freely within the interior of the outer protective sheath head, and the cutting bit instead advanced by holding the main body of the catheter relatively still while applying forward (distally directed) pressure on the torque communicating connector. By applying backward (proximally directed) pressure on the torque communicating connector, this process may also be reversed.

The outer protective sheath head may also contain a cavity through which a guidewire may be threaded from the proximal side of the sheath head through a distal opening in the sheath head. Often the distal opening used by the guidewire will be the same distal opening used by the cutting bit, but in some embodiments, the distal guidewire opening and the distal cutting bit opening can be two separate, or partially conjoined, openings.

Upon encountering an occlusion, the catheter can attempt to either traverse the occlusion, or at least insert the guidewire past the occlusion. In some embodiments, this may be done by an iterative process in which the cutting bit may be extended past the sheath head opening and rotated so as to cut or partially remove some of the occluding material. The cutting bit may then be retracted, and the cut occluding material then probed by extending the guidewire out through the sheath head and into the cut region. The guidewire can then be used to partially ream out or displace the cut occluding material. Depending upon the depth of the occluding material, the guidewire may then be retracted, the catheter sheath head advanced, the cutting bit extended, and further rounds of occluding material cutting can be performed, followed by further retraction of the cutting bit, further extension of the guidewire, and further reaming or probing of the cut material.

Once the operator has determined that the guidewire has successfully extended past the occlusion, the guidewire may then be extended further past the occlusion, and the catheter itself withdrawn. A second catheter, such as a balloon or alternative design atherectomy catheter may then be deployed up the guidewire and to the formerly occluded site. There the occlusion may be treated, for example by further enlargement, administration of therapeutic agents, stenting, or additional excision of occluding material, as needed.

Although, throughout this discussion, applications of this device for creating a passage through refractory atherosclerotic plaque from arteries, particularly coronary or peripheral limb arteries, are frequently used as examples, it should be understood that these particular examples are not intended to be limiting. Other applications for the present technology may include removal of kidney stones, in which case the device will be intended to traverse the ureters; gallstones, in which case the device will be intended to traverse the bile duct; enlarged prostate blockage of the urethra, in which case the device will be intended to traverse the urethra; blocked fallopian tubes, in which case the device will be intended to traverse the fallopian tubes; treatment of blood clots, removal of material trapped in the lungs, etc. In general, any unwanted material occupying space in a body lumen may be surgically removed by these techniques. Similarly, although use in human patients is cited in most examples, it should be evident that the same techniques may be useful in animals as well.

Helical drill bits and self-tapping screw bits are widely known to be highly effective at penetrating through materials as soft as wax and as refractory as rock and metal, and indeed such devices are widely used for such purposes. Although effective, drill bits are typically considered to be both powerful and extremely crude. As anyone who has ever attempted to use an electric drill can attest, drill devices, although admittedly effective at removing material, would seem to be totally unsuited for delicate vascular surgery, particularly at sites hidden deep within the body. Helical self-tapping screw bits are designed slightly differently. Although just as effective at cutting through various materials, drill bits are configured to both cut and then remove the material, while self-tapping screw bits are designed primarily for cutting a passage through the material. For either type of device, the problem is not the efficacy of cutting or occlusion removal; the problem is one of preventing inadvertent damage to the surrounding artery.

The invention provides a device, system and method that overcomes and obviates the prejudice against boring devices, and provides suitable protection and control for a type of “drill bit” device. Thus, now catheter “drill bit” devices configured according to the invention may now be suitable for delicate vascular surgery. Such a device would provide powerful solutions for cutting or removing occlusions, and yet configured to safely avoid unwanted damage to artery walls.

In one embodiment of the invention, the superior material cutting/removing properties of a material removal device are combined with suitable protection and catheter guidance mechanisms which allow such powerful cutting devices to be safely and effectively used within the confines of delicate arteries and other body lumens. One example is a self-threading helical screw bit configured to penetrate material within the body lumen.

To do this, precise control must be exerted over the cutting edge of the “drill bit”. The bit or “cutting head” should normally be sheathed or shielded from contact with artery walls, so that inadvertent damage to artery walls can be avoided while the head of the catheter is being threaded to the artery to the occluded region. Once at the occlusion, the cutting portion of the cutting head (bit) should be selectively exposed only to the minimal extent needed to perform the relevant occlusion cutting activity. The rotation direction of the cutting head may optionally be varied, for example by rotating the head counter-clockwise to produce a blunt dissection through the obstacle or occlusion, and then clockwise while pulling back on the entire assembly. Once the desired cuts are made, the cutting head should then be quickly returned to its' protective sheath. The entire device should operate within the millimeter diameters of a typical artery, and should be capable of being threaded on a catheter for a considerable distance into the body.

Suitable techniques to achieve these objectives are taught in the following figures and examples.

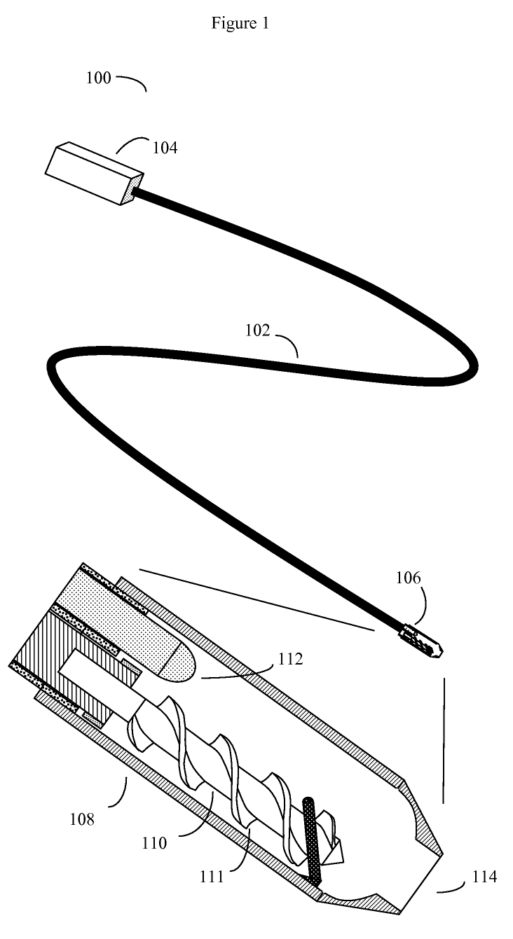

FIG. 1 shows an overview of one example of a catheter device (100) configured according to the invention that includes including the handle (104), the catheter (102), and the catheter sheath head (106). The catheter body and sheath head, are hollow and often have a cavity also capable of accommodating a guidewire. A magnified view of the catheter sheath head (108), showing the rotating cutting bit in a retracted configuration (110), the guidewire (112) inside of the sheath head guidewire cavity, the optional cutting bit blades (111), and the sheath head's distal opening (114) are also shown.

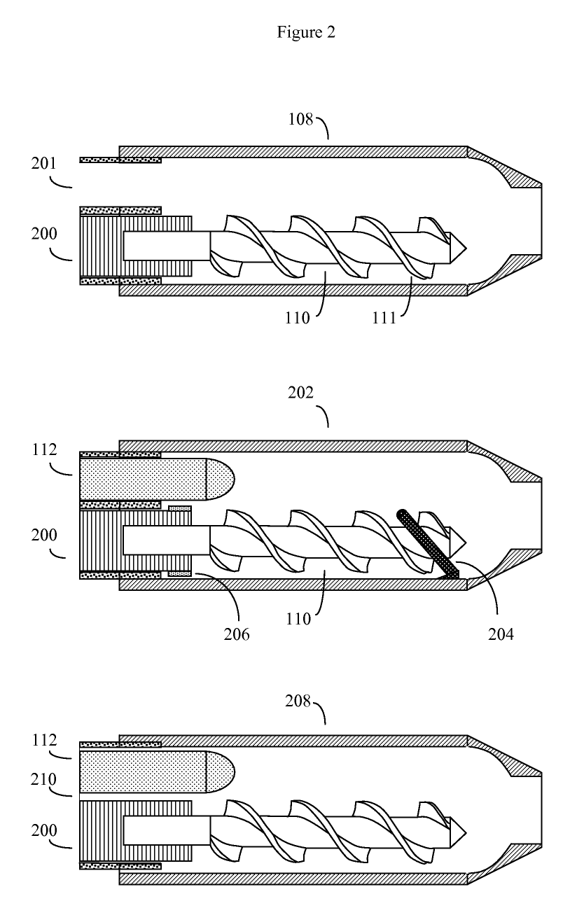

FIG. 2 shows various examples of alternate configurations of the interior of the catheter sheath head. In one configuration (108), the catheter contains a cavity (201) capable of admitting a guidewire into proximal end of the sheath head, and out of the distal opening or openings of the sheath head.

In some configurations, the guidewire (112) and the cutting bit drive cable (200) may travel though separate lumens in the catheter, and the motion of the cutting bit (110) may be otherwise not constrained by additional mechanisms inside the sheath head.

In other configurations (202), the motion of the cutting bit (110) may be guided (or rotary force directed into a linear force) by optional mechanisms such as a front annular device or mechanism (204). Motion constraint devices (motion stop devices) such as a rear bezel (206) may also be used. In a third configuration (208), the guidewire (112) and the cutting bit drive cable (200) travel though a common catheter lumen (210).

As previously discussed, the cutting bit (110) will optionally have projections (111), which may optionally have sharp cutting edges, in which case these edges will be referred to as blade edges. Often projections (111) will be helical blades, similar to the edges of a helical drill bit. Other configurations and non-helical blade or protrusion configurations may also be used, however.

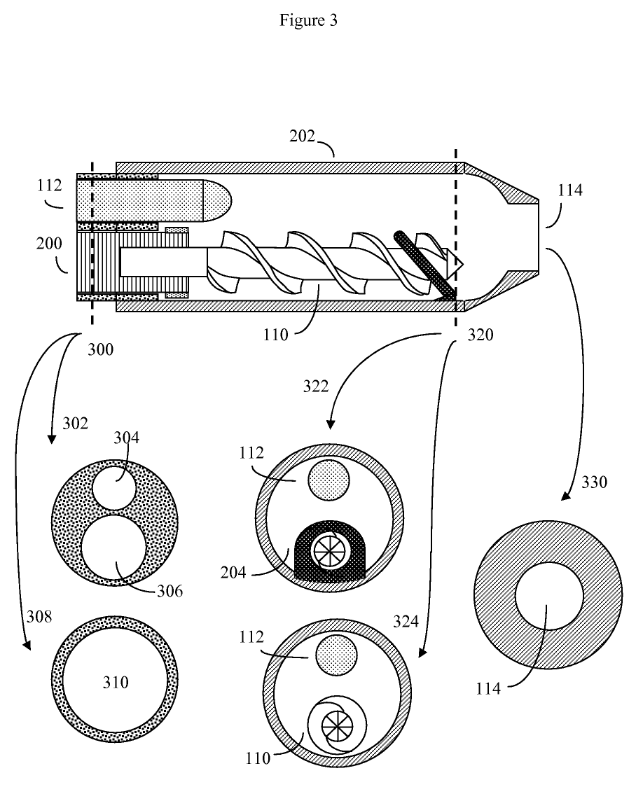

FIG. 3 shows various cross sections through the catheter tube and the catheter sheath head, and also shows various embodiments of the invention. In particular, various alternative sheath head (202) configurations are shown in more detail. Cross section (300), taken near where the body of the catheter extends into the proximal portion of the sheath head, shows that the catheter can contain either separate lumens for the guidewire and cutting bit drive cable (302), (304), (306), or alternatively (308) the catheter body can contain a single common lumen (310) where both the guidewire (112) and cutting bit drive cable (200) fit.

Cross section (320) shows a view from the distal portion of the sheath head at section (320) back towards the proximal portion of the catheter. As previously discussed, optionally there may be various types of mechanisms inside catheter sheath head (202) which can help direct the motion of the cutting bit and/or the guidewire. Example cross section (322) shows an embodiment where an optional mechanism (204) with a second surface interacts with a first surface (such as blades, slots, or protrusions) on cutting bit (110) and may help convert the circular motion of the cutting bit (110) supplied by torque from cable (200) into linear motion (either forward or backward) that can enable cutting bit (110) to protrude outside of the sheath head, through opening (114). By contrast, in an alternative embodiment (324), no such optional mechanism (204) need be present.

The tip of sheath head (202) is shown in an alternate (distal to proximal) view in (330). Here the opening (114) in the sheath head is shown in an alternate perspective.

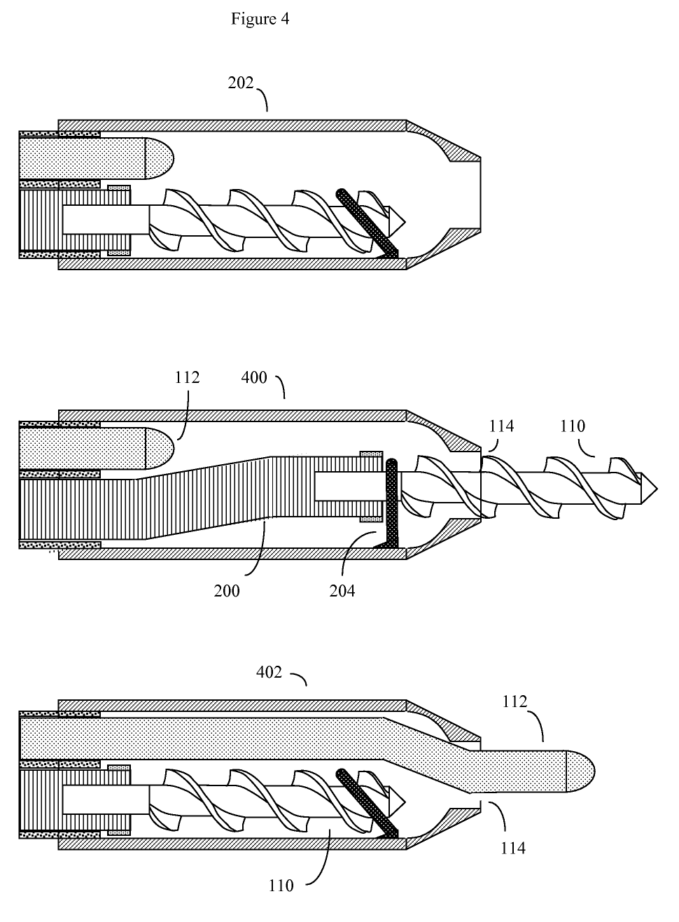

FIG. 4 shows how the cutting bit and the guidewire tip may be alternately extended and retracted through the opening at the distal tip of the catheter sheath head. In (202), both the cutting bit and the guidewire tip are fully retracted into the sheath head. In (400), the cutting bit (110) is partially extended outside the opening (114) of the sheath head (400), and drive cable (200) is also moved forward. In this drawing, optional mechanism (204) is also shown in a configuration where it has pivoted somewhat on a hinge or flexible support. Note that the guidewire tip (112) remains retracted.

In (402), the cutting bit (110) has again retracted inside the sheath head, and now the guidewire tip (112) has extended outside of the opening (114).

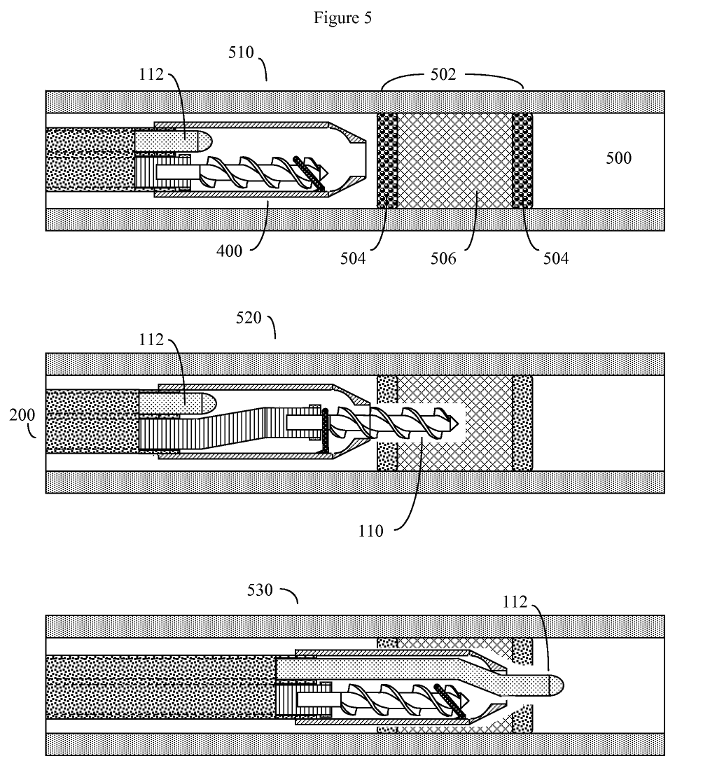

FIG. 5 shows use of the catheter in first cutting through the tough outer layer of a CTO obstruction, and then extending the guidewire through the CTO. Here a cross section of an artery (500) is shown. Inside the artery walls is a chronic total occlusion (CTO) (502) composed of tough outer end caps (504), and an inner layer which may be composed of atherosclerotic plaque, fibrous clot material, or other material (506).

In (510), the catheter and the catheter sheath head (400) are introduced up the artery to the CTO boundary, and then the guidewire (112) is partially retracted.

In (520), the cutting bit (110) is extended, torque is applied to the cutting bit drive cable (200), and the cutting bit (110) bores past the tough CTO end cap and partially into the CTO interior. The progress of this cutting operation may optionally be monitored or supplemented by withdrawing cutting bit (110), extending guidewire tip (112), and probing or reaming the cut CTO material with the guidewire tip.

In (530), possibly as a result of a number of cutting and probing/reaming operations, the cutting bit has cut through the second tough CTO outer layer. The guidewire (112) may now be fully extended past the CTO.

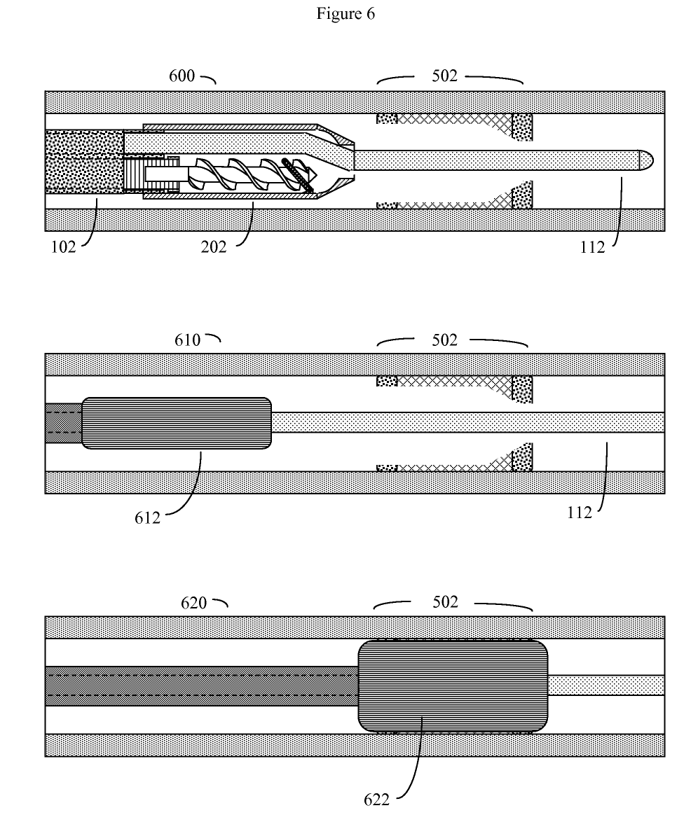

FIG. 6 can be viewed as a continuation of the CTO clearing process previously shown in FIG. 5, and shows how a balloon catheter or other device can then be introduced up the guidewire and to the CTO, where it then can be used to further treat the CTO. In (600), which is essentially a continuation of (530), the guidewire (112) has been further extended past the CTO (502), and eventually this guidewire may optionally be temporarily anchored in a second vascular opening or other structure. The catheter (102) and its sheath head (202) are shown in the process of being retracted away from the CTO (502).

In (610), the original catheter (102) and sheath head (202) have been totally withdrawn from the body, leaving just guidewire (112) remaining. A different catheter (612) (here portrayed as a balloon catheter), has then been threaded up the guidewire (112) to the site of the original and now partially unblocked CTO (502).

In (620), this different catheter (again portrayed as a balloon catheter) may be used to further treat the CTO. In this example, for ease of visualization, the balloon catheter (612) is shown both inflating (622) and further opening the sides of the CTO (502). Alternative treatments, such as administration of contrast agents, drugs, or other therapeutic agents may be done, and stenting or other alternative atherectomy procedures may also be done using the second catheter.

The sheath head portion of catheter head (106), (108) will normally be between about 1 to 2.2 millimeters in diameter, and the catheter body (102) will typically also have a diameter of approximately 1 to 3 millimeters (3-9 French), and a length between 50 and 200 cm. The sheath head may be made from various materials such as hard plastics, metals, or composite materials. Examples of such materials include NiTi steel, platinum/iridium or stainless steel.

Although sheath head (106), (108) may contain an optional inner mechanism (204) which may optionally contain a complementary second surface with slots, groves, annular structures or other features designed to impart forward motion to the first surface of cutting bit (110) when the cutting bit may be rotated. In general, the complementary second surface must be such that torque applied to the cutting bit causes the first surface on the cutting bit to engage the second surface, resulting in the cutting bit to both rotate and advance.

The cutting bit (110) will often be made of materials such as steel, carbide, or ceramic. The blades of the cutting head (111) can optionally be hardened by coating with materials such as tungsten carbide, ME-92, etc. Materials suitable for this purpose are taught in U.S. Pat. Nos. 4,771,774; 5,312,425; and 5,674,232. The angle of the blades and the details of their design will differ depending upon if the head is intended to simply cut through the occluding material, of if it is intended to cut through and actually remove (debulk) portions of the occlusion. For example, blades intended for to remove material may curve at an angle such that they will tend to sever the link between the occluding material and the body lumen, while blades intended just for cutting will have an alternate angle that tends not to sever this link.

In some embodiments, the catheter may be composed of two or more different tubes. In this illustrated configuration example, there may be an outer catheter tube (102), which will often be composed of a flexible biocompatible material. There may also be an inner connecting tube or cable (200) chosen for its ability to transmit torque from the catheter handle (104) to the cutting bit (110). The inner torque transmitting tube or cable (which may be one possible type of “torque communicating connector”) may be able to twist relative to the outer catheter tube so that when torque is applied to the inner tube or cable (200) at the handle end (104), the cutting bit (110) will rotate, but the catheter sheath head itself, which is connected to the outer catheter tube, will remain roughly stationary.

The outer catheter body (102) may often be made from organic polymer materials extruded for this purpose, such as polyester, polytetrafluoroethylene (PTFE), polyurethane, polyvinylchloride, silicon rubber, and the like. The inner torque conducting tube or cable (200) may be composed of these materials or alternatively may be composed from metal coils, wires, or filaments.

In many embodiments, the catheter will be designed with a cavity in the sheath head that allows a monorail guidewire (112) that has a diameter of about 0.014″, or between 0.010″ and 0.032″ into the proximal end of the sheath head, and out again by a distal opening in the sheath head. The catheter tube (102) will either contain a separate lumen for the guidewire, or alternatively have a common lumen where both the guidewire and the torque conducting tube or cable for the cutting bit.

In some embodiments, the guidewire may remain in the catheter body from the proximal base of the catheter up to the distal catheter sheath head. In other embodiments, the guidewire may reside outside of the catheter body for at least a portion of the length of the catheter, and then reenter the catheter body or the proximal end of the sheath head either near the distal end of the catheter, or alternatively some distance away, such as 10 cm, 20 cm, 30 cm, or more away from the distal end of the catheter.

In cases where the guidewire resides outside of the length of the catheter tube for a portion of the catheter length, the outer catheter jacket may optionally contain attached external guides for the monorail guidewire. In this case, the guidewire may exit these external guides either prior to the catheter head, or midway through the catheter head.

The catheter handle (104) will normally attach to both outer catheter tube (102), and inner tube or cable (200). Usually handle (104) will contain at least a knob, dial, or lever that allows the operator to apply torque to the inner torque transmitting tube or cable (200). In some embodiments, sensors may be used to determine how much the cutting bit (110) has rotated or extended relative to the sheath head portion of catheter head (106), and these sensors, possibly aided by a mechanical or electronic computation and display mechanism, may show the operator how much the cutting head has rotated and or extended.

In some embodiments, the catheter handle (104) will be designed with knobs or levers coupled to mechanical mechanisms (such as gears, torque communicating bands, etc.) that manually rotate and advance/retract the catheter tip, and the operator will manually control the tip with gentle slow rotation or movement of these knobs or levers. In other embodiments, the catheter handle may contain a mechanism, such as an electronic motor, and some type of controller, such as a button or trigger, that will allow the user to rotate and advance the cutting head in a precise and controlled manner. This mechanism may, for example, consist of a microprocessor or feedback controlled motor, microprocessor, and software that may act to receive information from a cutting head rotation or extension sensor, and use this rotation feedback data, in conjunction with operator instructions delivered by the button or trigger, to advance or retract the cutting head by a precise amount for each operator command. This way the operator need not worry about any errors induced by the spring action of the inner torque transmitting tube or cable (200). The microprocessor (or other circuit) controlled motor can automatically compensate for these errors, translate button or trigger presses into the correct amount of torque, and implement the command without requiring further operator effort. Alternatively non-microprocessor methods, such as a vernier or a series of guided markings, etc., may be used to allow the operator to compensate for differences in the rotation of the torque communicating connector and the rotation of the cutting head, or for the extent that which said cutting head exits said hollow sheath head.

In some embodiments, the catheter head may be equipped with additional sensors, such as ultrasonic sensors to detect calcified material, optical (near infrared) sensors to detect occlusions or artery walls, or other medically relevant sensors. If these sensors are employed, in some cases it may be convenient to locate the driving mechanisms for these sensors in the catheter handle (104) as well.

Additional features configured to improve the efficacy of the cutting bit and cutting head may also be employed. For example, the cutting bit or the cutting head may be configured to vibrate at high (ultrasonic) frequency, perform radiofrequency (RF) tissue ablation, generate localized areas of intense heat, conduct cutting light (e.g. laser or excimer laser), or other directed energy devices or systems.

The cutting bit may be composed of alternative designs and materials, and these designs and materials may be selected to pick the particular problem at hand. As an example, a cutting bit appropriate for use against a calcified obstruction may differ from the cutting bit appropriate for use against a non-calcified obstruction. Similarly the cutting bit appropriate for use against a highly fibrous obstruction may be less appropriate against a less fibrous and fattier obstruction. The length or size of the obstruction may also influence catheter sheath head and cutting bit design.

Although multiple catheters, each composed of a different type of cutting bit, may be one way to handle this type of problem, in other cases, a kit composed of a single catheter and multiple cutting bits (110) and optionally multiple sheath heads (106) may be more cost effective. In this type of situation, the cutting bits (110) may be designed to be easily mounted and dismounted from the drive cable or tube (200). A physician could view the obstruction by fluoroscopy or other technique, and chose to mount the cutting bit design (and associated sheath head design) best suited for the problem at hand. Alternatively, if the blades (111), on cutting bit (110) have become dull or chipped from use during a procedure, a physician may chose to replace dull or chipped cutting bit (110) with a fresh cutting bit, while continuing to use the rest of the catheter.

For some applications, it may also be useful to supply various visualization dyes or therapeutic agents to the obstruction using the catheter. Here, the dye or therapeutic agent may be applied by either sending this dye up to the catheter head through the space between the exterior catheter (102) and the interior torque tube or cable (200), or alternatively through a guidewire lumen, or separate liquid agent lumen, in catheter (102).

Examples of useful dyes and therapeutic agents to apply include fluoroscopic, ultrasonic, MRI, fluorescent, or luminescent tracking and visualization dyes, anticoagulants (e.g. heparin, low molecular weight heparin), thrombin inhibitors, anti-platelet agents (e.g. cyclooxygenase inhibitors, ADP receptor inhibitors, phosphodiesterase inhibitors, Glycoprotein IIB/IIIA inhibitors, adenosine reuptake inhibitors), anti-thromboplastin agents, anti-clot agents such as thrombolytics (e.g. tissue plasminogen activator, urokinase, streptokinase), lipases, monoclonal antibodies, and the like.

In some embodiments, it may be useful to construct the cutting bit out of a material that has a radiopaque signature (different appearance under X-rays) that differs from the material used to construct the hollow sheath head portion of the catheter head. This will allow the physician to directly visualize, by fluoroscopic or other x-ray imaging technique, exactly how far the cutting bit has advanced outside of the catheter sheath head.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope

1. A catheter comprising
a catheter tube with a proximal end, and a hollow distal end configured to enter a body lumen;
a cavity for a guidewire configured so that a guidewire with a proximal end and a distal tip inserted into the cavity is positionable within the hollow distal end of the catheter tube, and application of force on the proximal end of the guidewire causes the guidewire tip to extend forward at least partially outside of the distal end of the catheter tube; and
a distal rotatable cutting bit positionable within the hollow distal end of the catheter tube and configured to allow force applied to the rotatable cutting bit to cause the cutting bit to extend forward at least partially outside of the distal end of the catheter tube.

2. The catheter of claim 1, in which the application of distally directed linear force on the proximal end of a guidewire inserted into the cavity causes the guidewire tip to extend forward at least partially outside of the distal end of the catheter tube.

3. The catheter of claim 1, in which the application of proximally directed linear force on the proximal end of a guidewire inserted into the cavity causes a guidewire tip that has extended forward at least partially outside of the distal end of the catheter tube to retract back inside the distal end of the catheter tube.

4. The catheter of claim 1, in which torque force in a first direction applied to the rotatable cutting bit causes the rotatable cutting bit to extend forward at least partially outside of the distal end of the catheter tube.

5. The catheter of claim 1, in which torque force in a second direction applied to the rotatable cutting bit causes a rotatable cutting bit that has extended forward at least partially outside of the distal end of the catheter tube to retract back inside the distal end of the catheter tube.

6. The catheter of claim 1, in which the application of distally directed linear force on the rotatable cutting bit causes the rotatable cutting bit to extend forward at least partially outside of the distal end of the catheter tube.

7. The catheter of claim 1, in which the application of proximally directed linear force on the rotatable cutting bit causes a rotatable cutting bit that has extended forward at least partially outside of the distal end of the catheter tube to retract back inside the distal end of the catheter tube.

8. The catheter of claim 1, in which the cutting bit has at least one cutting edge.

9. The catheter of claim 1, in which a guidewire tip from a guidewire inserted into the cavity, and the cutting bit, exit the distal end of the catheter in the same location, and in which only one of the guidewire tip and the cutting bit may extend partially outside of the distal end of the catheter at the same time.

10. The catheter of claim 1, in which said catheter is used to remove material selected from the group consisting of refractory atherosclerotic plaque, chronic total occlusions from coronary arteries, and chronic total occlusions from peripheral limb arteries.

11. A catheter comprising a catheter tube with a proximal end, and a hollow distal end configured to enter a body lumen, a distal rotatable cutting bit, and a cavity for a guidewire with a proximal end and a distal tip;
said rotatable cutting bit and said cavity being configured so that a guidewire tip inserted into the cavity is capable of being positioned within the hollow distal end of the catheter tube;
said cavity and catheter tube being configured to allow application of distally and proximally directed reversible linear force on the proximal end of a guidewire inserted into the cavity to reversibly cause said guidewire tip to extend forward at least partially outside of the distal end of said catheter tube and to retract inside of the distal end of said catheter tube; and
said cutting bit and tube being configured to allow reversible torque force (torque) and optionally linear force reversibly applied to said rotatable cutting bit to cause the cutting bit to reversibly rotate and extend forward at least partially outside of the distal end of the catheter tube and to rotate and retract inside the distal end of the catheter tube.

12. The catheter of claim 11, in which the cutting bit has a first surface, and the distal end of the catheter tube has a complementary second surface configured to allow mechanical interactions between the cutting bit and the catheter tube to translate at least some reversible rotation of the cutting bit into extension of the cutting bit partially outside of the distal end of said catheter tube and to retraction inside the distal end of said catheter tube.

13. The catheter of claim 11, in which a guidewire tip inserted into the cavity and the cutting bit exit the distal end of the catheter in the same location, and in which only one of the guidewire tip and the cutting bit may extend partially outside of the distal end of the catheter at the same time.

14. The catheter of claim 11, in which the forward extension of the cutting bit is restricted by at least one motion stop element on the cutting bit.

15. The catheter of claim 11, wherein said catheter tube is hollow and contains a torque communicating connector, and wherein application of torque to said torque communicating connector causes said rotatable cutting bit to rotate.

16. The catheter of claim 15, wherein the torque is applied to the torque communicating connector by mechanisms selected from the group consisting of manual mechanisms, motors, and feedback controlled electronic motors.

17. The catheter of claim 15, wherein an agent selected from the group consisting of therapeutic agents and visualization dye agents is dispensed through the hollow catheter tube.

18. The catheter of claim 11, in which the cavity is configured so that at least a portion of a guidewire inserted into the cavity resides outside of the catheter for part of the proximal length of the catheter, and in which the guidewire then enters the distal portion of the catheter at least 20 centimeters back from the distal end of the catheter.

19. The catheter of claim 11, in which the cavity is configured so that a guidewire inserted into the cavity resides inside of the entire distal portion of the catheter designed to enter a body lumen.

20. The catheter of claim 11, wherein said rotatable cutting bit has at least one blade edge.

21. The catheter of claim 11, wherein the progress of the extension of said cutting bit outside of said catheter is monitored by a sensor.

22. The catheter of claim 11, wherein the cutting effectiveness of said cutting bit is enhanced by energy selected from the group consisting of ultrasonic vibration energy, radiofrequency (RF) energy, and light energy.

23. The catheter of claim 11, wherein the cutting bit has a radiopaque signature that is different from the radiopaque signature of the catheter.

24. The catheter of claim 11, in which said catheter is used to remove material selected from the group consisting of refractory atherosclerotic plaque, chronic total occlusions from coronary arteries, and chronic total occlusions from peripheral limb arteries.

25. The catheter of claim 11, in which said catheter is used as a pretreatment for a blocked artery, and in which the blocked artery is then subjected to further treatment selected from the group consisting of atherectomy, stenting and balloon angioplasty, following the use of said catheter.

26. A catheter comprising:
a catheter tube with a proximal end, and a distal end configured to enter a body lumen;
a hollow sheath head with a distal opening affixed at the distal end of said catheter tube;
a cavity for a guidewire with a proximal end and a distal tip, said cavity being configured so that a guidewire tip inserted into the cavity is capable of being positioned at least partially inside said hollow sheath head;
a rotatable cutting bit capable of being positioned at least partially inside said hollow sheath head;
said cavity, catheter tube, and hollow sheath head being configured to allow application of distally and proximally directed reversible linear force on the proximal end of a guidewire inserted into the cavity to reversibly cause said guidewire tip to extend forward at least partially outside of the distal opening of said hollow sheath head and to retract inside of the distal opening of said hollow sheath head;
said cutting bit, catheter tube, and hollow sheath head being configured to allow reversible torque force (torque) and optionally linear force reversibly applied to said rotatable cutting bit to cause the cutting bit to reversibly rotate and extend forward at least partially outside of the distal opening of said hollow sheath head and to rotate and retract inside the distal opening of said hollow sheath head.

27. The catheter of claim 26, in which the cutting bit has a first surface, and the interior of the hollow sheath head has a complementary second surface configured to allow mechanical interactions between the cutting bit and the interior of the hollow sheath head to translate at least some reversible rotation of the cutting bit into extension of the cutting bit partially outside of the distal opening of the hollow sheath head and to retraction inside the distal opening of the hollow sheath head.

28. The catheter of claim 27, in which the interior of the hollow sheath head contains an internal sheath head structure with an internal mechanism connected to the interior of the hollow sheath head;
the cutting bit protrudes through the internal mechanism;
and interactions between a first surface on the cutting bit and the internal mechanism translate at least some reversible rotation of the cutting bit into reversible extension of the cutting bit partially outside of the distal opening of the hollow sheath head and to retraction inside the distal opening of the hollow sheath head.

29. The catheter of claim 28, in which the internal mechanism is connected to the interior of the hollow sheath head by connectors selected from the group consisting of flexible supports and hinges.

30. The catheter of claim 26, in which a guidewire tip inserted into the cavity, and the cutting bit, exit the distal opening of the hollow sheath head in the same location, and in which only one of the guidewire tip and the cutting bit may extend partially outside of the distal opening of the hollow sheath head at the same time.

31. The catheter of claim 26, wherein said catheter tube is hollow and contains a torque communicating connector, and wherein application of torque to said torque communicating connector causes said rotatable cutting bit to rotate.

32. The catheter of claim 31, wherein the catheter tube contains a single hollow lumen, and the torque communicating connector and a guidewire inserted into this hollow lumen both reside inside this single hollow lumen for at least a portion of the catheter length.

33. The catheter of claim 31, wherein the catheter tube contains two hollow lumens, and in which the torque communicating connector resides inside a first hollow lumen, and a guidewire inserted into the second hollow lumen resides inside the second hollow lumen for at least a portion of the catheter length.

34. The catheter of claim 31, wherein the torque is applied to the torque communicating connector by mechanisms selected from the group consisting of manual mechanisms, motors, and feedback controlled electronic motors.

35. The catheter of claim 31, wherein an agent selected from the group consisting of therapeutic agents and visualization dye agents is dispensed through the hollow catheter tube.

36. The catheter of claim 31, in which at least a portion of a guidewire inserted into the cavity resides outside of the catheter for part of the proximal length of the catheter, and in which the guidewire then enters the distal portion of the catheter at least 20 centimeters back from the distal end of the catheter.

37. The catheter of claim 31, in which a guidewire inserted into the cavity resides inside the entire distal portion of the catheter designed to enter a body lumen.

38. The catheter of claim 26, wherein said rotatable cutting bit has at least one blade edge.

39. The catheter of claim 26, wherein the progress of the advancement of said cutting bit outside of said hollow sheath head opening is monitored by a sensor.

40. The catheter of claim 26, wherein the cutting effectiveness of said cutting bit is enhanced by energy selected from the group consisting of ultrasonic vibration energy, radiofrequency (RF) energy, and light energy.

41. The catheter of claim 26, wherein the cutting bit has a radiopaque signature that is different from the radiopaque signature of the hollow sheath head.

42. The catheter of claim 26, in which said catheter is used to remove material selected from the group consisting of refractory atherosclerotic plaque, chronic total occlusions from coronary arteries, and chronic total occlusions from peripheral limb arteries.

43. The catheter of claim 26, in which said catheter is used as a pretreatment for a blocked artery, and in which the blocked artery is then subjected to further treatment selected from the group consisting of atherectomy, stenting, and balloon angioplasty, following the use of said catheter.

44. A catheter comprising:
a hollow catheter tube with a proximal end, and a distal end configured to enter a body lumen;
a hollow sheath head with a distal opening affixed at the distal end of said catheter tube;
a cavity for a guidewire with a proximal end and a distal tip, said cavity being configured so that the distal tip of a guidewire inserted into the cavity can be positioned at least partially inside said hollow sheath head;
a rotatable cutting bit with at least one blade edge that is capable of being positioned at least partially inside said hollow sheath head;
and a torque communicating connector, wherein application of torque to said torque communicating connector causes said rotatable cutting bit to rotate;
said guidewire cavity, catheter tube, and hollow sheath head being configured to allow application of distally and proximally directed reversible linear force on the proximal end of a guidewire inserted into the cavity to reversibly cause said guidewire tip to extend forward at least partially outside of the distal opening of said hollow sheath head and to retract inside of the distal opening of said hollow sheath head;
said cutting bit, catheter tube, and hollow sheath head being configured to allow reversible torque force (torque) and optionally linear force reversibly applied to said rotatable cutting bit to cause the cutting bit to reversibly rotate and extend forward at least partially outside of the distal opening of said hollow sheath head and to rotate and retract inside the distal opening of said hollow sheath head;
in which a guidewire tip inserted into the cavity and the cutting bit exit the distal opening of the hollow sheath head in the same location, and in which only one of the guidewire tip and the cutting bit may extend partially outside of the distal opening of the hollow sheath head at the same time.

45. The catheter of claim 44, in which the cutting bit has a first surface, and the interior of the hollow sheath head has a complementary second surface configured to allow mechanical interactions between the cutting bit and the interior of the hollow sheath head to translate at least some reversible rotation of the cutting bit into extension of the cutting bit partially outside of the distal opening of the hollow sheath head and to retraction inside the distal opening of the hollow sheath head.

46. The catheter of claim 45, in which the interior of the hollow sheath head contains an internal sheath head structure with an internal mechanism connected to the interior of the hollow sheath head;
the cutting bit protrudes through the internal mechanism;
and interactions between a first surface on the cutting bit and the internal mechanism translate at least some reversible rotation of the cutting bit into reversible extension of the cutting bit partially outside of the distal opening of the hollow sheath head and to retraction inside the distal opening of the hollow sheath head.

47. The catheter of claim 46, in which the internal mechanism is connected to the interior of the hollow sheath head by connectors selected from the group consisting of flexible supports and hinges.

48. The catheter of claim 44, wherein the catheter tube contains a single hollow lumen, and the torque communicating connector and a guidewire inserted into the lumen both reside inside this single hollow lumen for at least a portion of the catheter length.

49. The catheter of claim 44, wherein the catheter tube contains two hollow lumens, and in which the torque communicating connector resides inside a first hollow lumen, and the a guidewire inserted into the second hollow lumen resides inside the second hollow lumen for at least a portion of the catheter length.

50. The catheter of claim 44, wherein the torque is applied to the torque communicating connector by mechanisms selected from the group consisting of manual mechanisms, motors, and feedback controlled electronic motors.

51. The catheter of claim 44, wherein an agent selected from the group consisting of therapeutic agents and visualization dye agents is dispensed through the hollow catheter tube.

52. The catheter of claim 44, in which at least a portion of a guidewire inserted into the cavity resides outside of the catheter for part of the proximal length of the catheter, and in which the guidewire then enters the distal portion of the catheter at least 20 centimeters back from the distal end of the catheter.

53. The catheter of claim 44, in which a guidewire inserted into the cavity resides inside the entire distal portion of the catheter designed to enter a body lumen.

54. The catheter of claim 44, wherein the progress of the advancement of said cutting bit outside of said hollow sheath head opening is monitored by a sensor.

55. The catheter of claim 44, wherein the cutting effectiveness of said cutting bit is enhanced by energy selected from the group consisting of ultrasonic vibration energy, radiofrequency (RF) energy, and light energy.

56. The catheter of claim 44, wherein the cutting bit has a radiopaque signature that is different from the radiopaque signature of the hollow sheath head.

57. The catheter of claim 44, in which said catheter is used to remove material selected from the group consisting of refractory atherosclerotic plaque, chronic total occlusions from coronary arteries, and chronic total occlusions from peripheral limb arteries.

58. The catheter of claim 44, in which said catheter is used as a pretreatment for a blocked artery, and in which the blocked artery is then subjected to further treatment selected from the group consisting of atherectomy, stenting, and balloon angioplasty following the use of said catheter.
